(19) United States
(12) Patent Application Publication
(10) Pub. No.: US 2011/0091396 A1
(43) Pub. Date: Apr. 21, 2011

(54) SUNLESS TANNING COMPOSITION INSURING EFFECTIVE APPLICATION TO SKIN

(75) Inventors: Lei Huang, Trumbull, CT (US); Qiang Qiu, Trumbull, CT (US); Zhi-xing Jiang, Southbury, CT (US); Jeffrey William Rosevear, Wallingford, CT (US)

(73) Assignee: CONOPCO, INC., D/B/A UNILEVER, Englewood Cliffs, NJ (US)

(21) Appl. No.: 12/580,481

(22) Filed: Oct. 16, 2009

(51) Int. Cl. A61K 8/33; A61Q 19/04

(52) U.S. Cl. 424/59

(57) ABSTRACT

A sunless tanning composition is provided with from 0.1 to 40% of a C₁-C₂₄ alpha-hydroxyaldehyde and from 0.1 to 15% of hydrophobic droplets, the droplets including from 80 to 99.9% of a lipophilic substance and from 0.01 to 3% of a particulate colorant dispersed within the lipophilic substance, and wherein upon initial application to an area of skin the droplets appear aesthetically displeasingly grey/black which upon being rubbed into the skin dissipates the aesthetically displeasing grey/black appearance and concurrently assures sufficient rub-in of the alpha-hydroxyaldehyde to evenly and penetratingly interact with the area of skin.

### BACKGROUND OF THE INVENTION

1. Field of the Invention

The invention concerns a composition which efficiently delivers a sunless tan to skin.

2. The Related Art

Today there is a great health concern with natural tanning through sunlight. Ultraviolet radiation from the sun is considered to be a leading factor in causing skin cancer. Even if not, lethal, ultraviolet radiation has been acknowledged as accelerating aging and wrinkling processes on the skin.

Beyond health concerns, there are obvious practical reasons against natural tanning. Foremost is the reason that in many areas of the globe and during all but summertime, there is insufficient sunlight available to accomplish a natural tan.

Based on the above considerations, there has been much interest in effectuating a tan through cosmetic means. Dihydroxyacetone (hereinafter also known as DHA) has widely been utilized in cosmetics to accomplish self-tanning of the skin. See the J. Soc. Cosmet. Chem., 35, pages 265-272 (August 1984).

U.S. Pat. No. 5,612,044 (Suares et al.) employs a multi-compartment dispenser to deliver the cosmetic. In a first of the separate compartments is a combination of dihydroxyacetone and a silicone. A second of the compartments stores a substance containing at least one amino acid. Upon being simultaneously dispensed from their separate compartments, amino acids from the second substance combine with the DHA from the first substance to initiate a browning reaction on the skin. Control of dispensing is difficult with multi-compartment release and often results in uneven application.

A similar idea is described in WO 97/33560 (Meyer et al.) where the two actives substances are DHA and, instead of an amino acid, a secondary polyamine. An expensive and difficult to control multi-compartment dispenser likewise is necessary for this approach. Unevenness of application leads to a streaky tan.

A number of problems are commonly encountered in sunless tanning systems. Streakiness in color development is one of the greatest issues. Uneven application is a primary cause of streaky browning of the skin. Better solutions are needed for this and other issues in developing an artificial tan.

### SUMMARY OF THE INVENTION

A sunless tanning composition is provided with from 0.1 to 40% of a C₁-C₂₄ alpha-hydroxyaldehyde and from 0.1 to 15% of hydrophobic droplets, the droplets including from 80 to 99.9% of a lipophilic solid substance and from 0.01 to 3% of a particulate colorant dispersed within the lipophilic substance, and wherein upon initial application to an area of skin the droplets appear aesthetically displeasingly grey/black which upon being rubbed into the skin dissipates the aesthetically displeasingly grey/black appearance and concurrently assures sufficient rub-in of the alpha-hydroxyaldehyde to evenly and penetratingly interact with the area of skin.

### DETAILED DESCRIPTION OF THE INVENTION

Now there has been provided a sunless tanning composition with a mechanism to insure a more thorough and even rub-in over an area of skin. Continuous repeated rub-in allows the sunless tanning agent (e.g. dihydroxyacetone) component of the composition to be evenly spread and driven into the skin. The desired user behavior of a thorough and even rub-in with application of finger pressure results from presence of dark colored discrete iron oxide particles suspended in fracturable hydrophobic droplets or beads. Initial pressure against the composition causes the droplets to break up and disperse the iron oxide particles leaving a displeasingly grey/black color on the skin. In an effort to rub-out the ugly and now streaky grey/black color, the consumer unwittingly rubs the sunless tanning agent present in the formula more broadly and more evenly over the skin. Applicant has found that the rub-in to rub-out the aesthetically displeasing color eliminates any eventual sunless tan streaking normally caused by an uneven application.

Accordingly, an important element of the present invention is that of hydrophobic droplets within the composition. Amounts of the droplets may range from 0.1 to 15% by weight, preferably from 0.5 to 10%, more preferably from 1 to 5%, and optimally from 1.5 to 3% by weight of the composition. Average droplet size may range from 50 to 3,000 micron, preferably from 100 to 1500 micron, more preferably from 15 to 1200 micron, and optimally from 200 to 900 micron.

Each of the hydrophobic droplets will contain a lipophilic substance operative as a carrier matrix for a particulate colorant. Useful lipophilic substances may include petrolatum, silicones, glycerides and combinations thereof. Glycerides may be di- and tri-glycerides. Illustrative triglycerides are sunflower seed oil, cottonseed oil, soybean oil and safflower oil. Most preferred as the lipophilic substance is petrolatum. Amounts of the semi-solid lipophilic substances may range from 80 to 99.9%, preferably from 95 to 99%, and optimally from 95 to 98% by weight of the hydrophobic droplet.

A particulate colorant will be embedded within the lipophilic substance. The colorant may be an organic dye or lake but, more preferably, is a water-insoluble pigment. Illustrative pigments include chromium oxides, iron oxides, cobalt salts, charcoal and mixtures thereof. Most preferred is iron oxide. Advantageously the iron oxide is black. In preferred embodiments, additional iron oxide of red and yellow color also are present. For these embodiments the relative weight ratio of black to red may range from 2:1 to 1:2, preferably from 1.5:1 to 1:1. The weight ratio of black to yellow may range from 6:1 to 1:1, preferably from 4:1 to 2:1, optimally from 3:1 to 2.2:1.

The particulate colorant is dispersed within the lipophilic substance. Amounts of the total colorant may range from 0.01 to 3%, preferably from 0.05 to 2%, more preferably from 0.1 to 1%, and optimally from 0.3 to 0.8% by weight of the hydrophobic droplet.

In a preferred embodiment, the lipophilic substance is formed from petrolatum in which is embedded black iron oxide particulates.

Another important element of the present invention is that of a sunless tanning agent. This agent will be a C₁-C₂₄ alpha-hydroxyaldehyde. The amount of this material will range from 0.1 to 40%, preferably from 1 to 20%, optimally between 2 and 5% by weight. The alpha-hydroxy aldehyde may be selected from dihydroxyacetone, glucose, xylose, fructose, reose, ribose, pentose, arabinose, allose, tallose, altrose, mannose, galactose, lactose, sucrose, erythrose, glyceraldehydes and combinations thereof. Most preferred is dihydroxyacetone.

Compositions of this invention may include structuring systems. In one embodiment, the structuring system may be a crystalline gel structurant. For this embodiment, the structurant may include both a surfactant and a co-surfactant. Preferred surfactants are C₁-C₂₀₀ esters of C₁₀-C₂₂ fatty acid. Esters of the fatty acid preferably are polyol esters such as C₂-C₃ alkoxylated alcohol esters. Among these are the polyethoxy, polypropoxy and block polyethyoxy/polypropoxy alcohol esters. Particularly preferred are such esters as PEG-100 stearate, PEG-20 stearate, PEG-80 laurate, PEG-20 laurate, PEG-100 palmitate, PEG-20 palmitate and combinations thereof.

The co-surfactant typically may be a combination of a C₁₀-C₂₂ fatty alcohol, glyceryl esters of a C₁₀-C₂₂ fatty acid, and a C₁₀-C₂₂ unesterified fatty acid. Relative amounts of the ester to the alcohol may range from about 100:1 to about 1:100, preferably from about 50:1 to about 1:50, and optimally from about 3:1 to about 1:3 by weight. Relative amounts of the combination of glyceryl ester and fatty alcohol to unesterified fatty acid may range from about 100:1 to about 1:100, preferably from about 50:1 to about 1:50, and optimally from about 3:1 to about 1:3 by weight. Typical fatty alcohols include behenyl alcohol, stearyl alcohol, cetyl alcohol, myristyl alcohol, lauryl alcohol, oleyl alcohol and combinations thereof.

The relative amount of surfactant and co-surfactant may range from about 50:1 to about 1:50, preferably from about 10:1 to about 1:10, and optimally from about 3:1 to about 1:3 by weight.

Advantageously, the crystalline gel structurant may constitute from 50 to 99%, preferably from 60 to 95%, and optimally from 75 to 90% by weight of the composition. The preferred embodiment of the crystalline gel structurant may comprise from 0.1% to 20% of stearic acid, from 0.1% to 20% of glycol or glycerol stearate, and from 0.1% to 20% of PEG stearate.

Compositions of the present invention are not limited by any pH range. However, a preferred pH ranges from about 3.5 to about 7, preferably from about 3.5 to about 6, and optimally around 4.5.

Thickeners may be present. Illustrative thickeners are the chemically modified starches such as sodium hydroxypropyl starch phosphate available from the National Starch and Chemical Company and from Grain Processing Corporation, the latter under the trademark Pure-Gel®. Polymers such as taurates may be useful as thickeners. One example is hydroxyethyl acrylate/sodium acryloyldimethyl taurate copolymer. By the term thickener is meant any material which at 2% in water generates a viscosity greater than 5,000 cps, particularly greater than 10,000 cps as measured on a Brookfield RVT model viscometer at spindle speed of 20 rpm in a pH range 7.2 to 7.6.

A variety of other components may be present in the compositions of the present invention. Foremost is that of water. Amounts of water may range from about 1 to about 90%, preferably from about 30 to about 80%, optimally from about 50 to about 80% by weight.

Cosmetic compositions of the present invention may include glycerin for moisturization. Amounts of glycerin when present may range from about 1% to about 50%, preferably from 5% to 40%, more preferably from 8 to 35%, optimally from 10 to 30% by weight of the composition.

Emollient materials may be included in compositions of this invention. These may be in the form of silicone oils, synthetic esters and hydrocarbons. Amounts of the emollients may range anywhere from about 0.1 to about 95%, preferably between about 1 and about 50% by weight.

Silicone oils may be divided into the volatile and nonvolatile variety. The term “volatile” as used herein refers to those materials which have a measurable vapor pressure at ambient temperature. Volatile silicone oils are preferably chosen from cyclic (cyclomethicone) or linear polydimethylsiloxanes containing from 3 to 9, preferably from 4 to 5, silicon atoms.

Nonvolatile silicone oils useful as an emollient material include polyalkyl siloxanes, polyalkylaryl siloxanes and polyether siloxane copolymers. The essentially nonvolatile polyalkyl siloxanes useful herein include, for example, polydimethyl siloxanes with viscosities of from about 5×10⁻⁶ to 0.1 m²/s at 25° C. Among the preferred nonvolatile emollients useful in the present compositions are the polydimethyl siloxanes having viscosities from about 1×10⁻⁵ to about 4×10⁻⁴ m²/s at 25° C.

Another class of nonvolatile silicones are emulsifying and non-emulsifying silicone elastomers. Representative of this category is Dimethicone/Vinyl Dimethicone Crosspolymer available as Dow Corning 9040, General Electric SFE 839, and Shin-Etsu KSG-18. Silicone waxes such as Silwax WS-L (Dimethicone Copolyol Laurate) may also be useful.

Among the ester emollients are:
(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C₁-C₃₀ alcohols.
(4) Wax esters such as beeswax, spermaceti wax and tribehenin wax.
(5) Sterols esters, of which cholesterol fatty acid esters are examples thereof.
(6) Sugar ester of fatty acids such as sucrose polybehenate and sucrose polycottonseedate.

Hydrocarbons are suitable further ingredients. These include petrolatum, mineral oil, C₁₁-C₁₃ isoparaffins, polyalphaolefins, and especially isohexadecane, available commercially as Permethyl 101A from Presperse Inc.

Humectants of the polyhydric alcohol-type in addition to glycerin can be employed with formulations of this invention. Typical polyhydric alcohols include polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. The amount of humectant may range anywhere from 0.5 to 50%, preferably between 1 and 15% by weight of the composition.

Sunscreen actives may also be included in compositions of the present invention. Particularly preferred are such materials as ethylhexyl p-methoxycinnamate, available as Parsol MCX®, Avobenzene, available as Parsol 1789® and benzophenone-3, also known as Oxybenzone. Inorganic sunscreen actives may be employed such as microfine titanium dioxide, zinc oxide, polyethylene and various other polymers. Amounts of the sunscreen agents when present may generally range from 0.1 to 30%, preferably from 2 to 20%, optimally from 4 to 10% by weight.

Preservatives can desirably be incorporated into the cosmetic compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients in the emulsion. Preservatives are preferably employed in amounts ranging from 0.01% to 2% by weight of the composition.

Compositions of the present invention may also contain vitamins. Illustrative water-soluble vitamins are Niacinamide, Vitamin B₂, Vitamin B₆, Vitamin C and Biotin. Among the useful water-insoluble vitamins are Vitamin A (retinol), Vitamin A Palmitate, Ascorbyl Tetraisopalmitate, Vitamin E (tocopherol), Vitamin E Acetate and DL-panthenol. Anti-Aging actives may also be present including flavanoids, resveratrol and epidihydroandrosterol (DHEA). Total amount of vitamins and anti-aging actives when present in compositions according to the present invention may range from 0.001 to 10%, preferably from 0.01% to 1%, optimally from 0.1 to 0.5% by weight.

Desquamation agents are further optional components. Illustrative are the alpha-hydroxycarboxylic acids and beta-hydroxycarboxylic acids. Among the former are salts of glycolic acid, lactic acid and malic acid. Salicylic acid is representative of the beta-hydroxycarboxylic acids. Amounts of these materials when present may range from about 0.1 to about 15% by weight of the composition.

A variety of herbal extracts may optionally be included in compositions of this invention. Illustrative are green tea, white tea, chamomile, licorice and extract combinations thereof. The extracts may either be water soluble or water-insoluble carried in a solvent which respectively is hydrophilic or hydrophobic. Water and ethanol are the preferred extract solvents.

Colorants, fragrances, opacifiers and abrasives may also be included in compositions of the present invention. Each of these substances may range from about 0.05 to about 5%, preferably between 0.1 and 3% by weight.

Except in the operating and comparative examples, or where otherwise explicitly indicated, all numbers in this description indicating amounts of material ought to be understood as modified by the word “about”.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

All documents referred to herein, including all patents, patent applications, and printed publications, are hereby incorporated by reference in their entirety in this disclosure.

The following examples will more fully illustrate the embodiments of this invention. All parts, percentages and proportions referred to herein and in the appended claims are by weight unless otherwise illustrated.

### Example 1-8

Illustrative formulas of the present invention are those outlined in Table I.

TABLE I

| | Weight % | | | | | | | |
|---|---|---|---|---|---|---|---|---|
| Ingredient | 1 | 2 | 3 | 4 | 5 | 6 | 7 | 8 |
| Glycerin | 8.00 | 12.00 | 12.00 | 5.00 | 5.00 | 5.00 | 8.00 | 8.00 |
| Isopropyl Palmitate | 4.50 | 4.50 | 4.50 | 3.50 | 3.50 | 3.50 | 6.50 | 4.50 |
| Glycerol Stearate/ | 3.00 | 3.25 | 2.50 | 2.00 | 2.00 | 3.00 | 1.50 | 2.50 |
| Stearamide AMP | | | | | | | | |
| Dihydroxyacetone | 2.50 | 1.50 | 2.50 | 2.00 | 2.00 | 2.00 | 1.00 | 3.50 |
| PEG-100 Stearate | 2.00 | 2.00 | 2.00 | 2.00 | 2.00 | 2.00 | 2.00 | 2.00 |
| Stearic Acid | 2.00 | 2.00 | 2.00 | 2.00 | 2.00 | 2.00 | 2.00 | 2.00 |
| Simulgel NS ®* | 1.50 | 1.50 | 1.50 | 1.50 | 1.50 | 1.50 | 1.50 | 1.50 |
| Glyceryl Stearate | 1.10 | 1.10 | 1.10 | 1.10 | 1.10 | 1.10 | 1.10 | 1.10 |
| Dimethicone | 1.00 | 1.00 | 1.00 | 1.00 | 1.00 | 1.00 | 1.00 | 1.00 |
| Cetyl Alcohol | 0.60 | 0.60 | 0.60 | 0.60 | 0.60 | 0.60 | 0.60 | 0.60 |
| Cyclopentasiloxane/ | 0.50 | 0.50 | 0.50 | 0.50 | 0.50 | 0.50 | 0.50 | 0.50 |
| Dimethiconol | | | | | | | | |
| Titanium Dioxide | 0.50 | 0.50 | 0.50 | 0.50 | 0.50 | 0.50 | 0.50 | 0.50 |
| Phenoxyethanol | 0.40 | 0.40 | 0.40 | 0.40 | 0.40 | 0.40 | 0.40 | 0.40 |
| Fragrance | 0.40 | 0.40 | 0.40 | 0.40 | 0.40 | 0.40 | 0.40 | 0.40 |
| Methylparaben | 0.20 | 0.20 | 0.20 | 0.20 | 0.20 | 0.20 | 0.20 | 0.20 |
| Citric Acid | 0.11 | 0.11 | 0.11 | 0.11 | 0.11 | 0.11 | 0.11 | 0.11 |
| Tetrasodium EDTA | 0.11 | 0.11 | 0.11 | 0.11 | 0.11 | 0.11 | 0.11 | 0.11 |
| Sodium Hydroxypropyl | 0.10 | 0.10 | 0.10 | 0.10 | 0.10 | 0.10 | 0.10 | 0.10 |
| Starch Phosphate | | | | | | | | |
| Propyl Paraben | 0.10 | 0.10 | 0.10 | 0.10 | 0.10 | 0.10 | 0.10 | 0.10 |
| Water | Bal | Bal | Bal | Bal | Bal | Bal | Bal | Bal |
| Droplets: | | | | | | | | |
| Petrolatum | 3.00 | 3.00 | 2.50 | 3.50 | 2.50 | 3.50 | 4.00 | 5.00 |
| Iron Oxide Red | 0.25 | 0.25 | 0.25 | 0.25 | 0.25 | 0.25 | 0.25 | 0.25 |
| Iron Oxide Yellow | 0.10 | 0.10 | 0.10 | 0.10 | 0.10 | 0.10 | 0.10 | 0.10 |
| Iron Oxide Black | 0.80 | 0.50 | 0.25 | 0.35 | 1.25 | 0.30 | 0.40 | 1.25 |

*Hydroxyethyl Acrylate/Sodium Acryloyldimethyltaurate copolymer

The compositions recorded in Examples 1-8 may be prepared in the following manner. Water is charged to a batching vessel. Mixing is begun at about 250 rpm with heating to 75-80°. Simulgel NS® and the starch phosphate are slowly added into the water. Mixing speed is increased slightly where necessary, making sure to avoid clumping. All other water soluble ingredients are then added. In a separate vessel, oily phase ingredients are combined. Initially there is light mixing of the oily phase with heat applied to achieve 75-80° C. temperature. In still a further vessel, petrolatum and the iron oxides are added. This vessel is heated to 55° C. until uniform distribution of the components. When the temperature of both water and oily phases have reached 80° C., the oily phase is slowly added under moderate mixing (270 rpm) to the water phase. The combination is then homogenized for 1 minute. Temperature is then allowed to cool to 45° C. and dihydroxyacetone in water is added. The resultant mixture is then cooled to 40° C. whereupon the silicones, phenoxyethanol and fragrance is added to the batch. At 35° C., the mixture is caused to sweep at 200 rpm. Thereafter the petrolatum premixture (at 55° C.) is slowly added to the combined water/oily phases in a manner to insure droplet formation. Speed of mixing is increased to obtain uniform dispersion of the petrolatum droplets throughout the composition.

A sample of the resultant formula can be applied to a treatment area of the body. The composition is slowly rubbed into the skin. Aesthetically displeasing black streakish color is seen as the petrolatum droplets are broken releasing the black iron oxide. User may rub anywhere from 5 seconds to 10 minutes, preferably from 10 seconds to 2 minutes, and optimally from 0.5 to 2 minutes till the composition has been sufficiently dispersed over the skin treatment area so that any of the black iron oxide can no longer be perceived.

### Example 9

Experiments under this Example demonstrate the concept of the present invention. The formula according to Example 3 was compared to an identical formula except with the absence of any iron oxides from the petrolatum droplets (control).

Protocol for the test was as follows. A subject was required to apply both formulas on fixed sites of their arm in the morning and at night for three days. The subject was then advised to rub-in the formula to the point that they felt was sufficient for having evenly applied same onto skin. Following the third day's formula application, images of both application sites were taken and streakiness of images was evaluated.

Measurement of streakiness was done in the following manner. After formula application, a standard photograph of the skin was taken. The region of interest was cropped for further image analysis. An algorithm was utilized to extract the area of streakiness. The algorithm involved use of a Gaussian calculation at pre-selected filter size of 6 and 35. The area of streakiness was then obtained by subtracting the two Gaussian filtered images. Streakiness was measured as the ratio of the sum of the streakiness area to total area. The greater the resultant value, the greater the streakiness. Thus, lower ratio values indicate improved uniformity and less streakiness.

TABLE II

| Formula | Description | Streakiness Ratio |
|---|---|---|
| 1 (inventive) | DHA + iron oxide in petrolatum | 0.25 |
| | droplets | |
| 2 (control) | DHA + clear petrolatum droplets | 0.29 |

* DHA is dihydroxyacetone

Based on the panelist testing, it is evident that the iron oxide/petrolatum droplets formula of the present invention achieved a decrease in streakiness. The ratio of Formula 1 was only 0.25 compared with the higher value of 0.29 for an identical Formula 2 that did not include iron oxide particulates. It is evident that the displeasing aesthetics of the dark colored iron oxide particulates caused the panelists to more evenly distribute sample over the application area.

What is claimed is:

1. A sunless tanning composition comprising from 0.1 to 40% of a C₁-C₂₄ alpha-hydroxyaldehyde and from 0.1 to 15% of hydrophobic droplets, the droplets comprising from 80 to 99.9% of a lipophilic substance and from 0.01 to 3% of a particulate colorant dispersed within the lipophilic substance, and wherein upon initial application to the skin the droplets appear aesthetically displeasingly grey/black which upon being rubbed into an area of skin dissipates the aesthetically displeasingly grey/black appearance and concurrently assures sufficient rub-in of the alpha-hydroxyaldehyde to evenly and penetratingly interact with the area of skin.

2. The composition according to claim 1 wherein the lipophilic substance is selected from a group consisting of petrolatum, silicones, glycerides and mixtures thereof.

3. The composition according to claim 2 wherein the lipophilic substance is petrolatum.

4. The composition according to claim 1 wherein the colorant comprises black iron oxide.

5. The composition according to claim 1 wherein the alpha-hydroxyaldehyde is dihydroxyacetone.

6. The composition according to claim 1 wherein the hydrophobic droplets have an average size ranging from 50 to 3,000 micron.

7. The composition according to claim 1 wherein the hydrophobic droplets have an average size ranging from 200 to 900 micron.

8. The composition according to claim 1 which is a crystalline gel structurant and comprises from 0.1% to 20% of stearic acid, from 0.1% to 20% of glycol or glycerol stearate, and from 0.1% to 20% of PEG stearate.
